(19) United States
(12) Patent Application Publication
(10) Pub. No.: US 2026/0217637 A1
(43) Pub. Date: Jul. 30, 2026

(54) SYSTEMS AND METHODS FOR PRODUCTION OF ACRYLIC ACID

(71) Applicant: Lummus Technology LLC, Bloomfield, NJ (US)

(72) Inventors: Abhishek AGRAWAL, Gurugram (IN); Shaun MCGOVERN, Glen Rock, NJ (US); Sunil PANDITRAO, Budd Lake, NJ (US); Sanjeev RAM, Berkeley Heights, NJ (US)

(21) Appl. No.: 19/040,112

(22) Filed: Jan. 29, 2025

(51) Int. Cl. C07C 51/25; B01J 12/00; B01J 19/00

(52) U.S. Cl. CPC C07C 51/252; B01J 12/00; B01J 19/0013; B01J 2219/00074; B01J 2219/00159

(57) ABSTRACT

Systems and methods for the production of acrylic acid include vaporizing a propylene feed and separately compressing an air stream. The compressed air may be mixed with steam or recycle gas, or both. The vaporized propylene and the compressed air mixture are combined and mixed at a mixer to produce a reaction feed. The reaction feed is mixed at a lower temperature than the reaction temperature of the reaction feed to improve safety. The reaction feed is heated to the reaction temperature by a heat exchanger downstream of the mixer. The heating medium in the heat exchanger may be steam to control the heating process and further improve safety.

### BACKGROUND

### Technical Field

The present disclosure is generally directed to systems and methods for production of acrylic acid.

### Description of the Related Art

Acrylic acid is a chemical intermediate used in a variety of industries and products, such as in water treatment, super absorbent polymers, textiles, diapers, paint, and others. The most common process for making acrylic acid is the vapor phase oxidation of propylene. In conventional processes for producing acrylic acid, propylene vapors are mixed with air, steam and recycle gas before being fed to two reactors in series. The temperature of the air, steam and recycle gas mixture is in the range of 120-220°C, and preferably in the range 160-200°C. The propylene vapors have a temperature in a range of 50-75°C. The resultant propylene, air, steam and recycle gas mixture provided to the reactors has a temperature in a range of 140-180°C. At the first reactor, propylene is oxidized to acrolein and at the second reactor, acrolein is oxidized to acrylic acid.

Propylene is highly flammable such that when it is mixed with hot air in conventional processes, the mixing and/or mixture will pass through the flammability envelope. Mixtures of combustible materials and oxygen in the air will burn only if the fuel concentration (relative to oxygen) lies within lower and upper bounds known as lower and upper flammability limits, respectively. The lower flammability limit (“LFL”) is the lowest concentration percentage of a gas or a vapor in air capable of producing a flash of fire in the presence of an ignition source. If the concentration of gas is below the LFL, the gas mixture is too lean to ignite and burn. The upper flammability limit (“UFL”) is the highest concentration of a gas or a vapor in air capable of producing a flash of fire in the presence of an ignition source. Concentrations higher than the UFL are too rich to ignite and burn. The LFL and UFL for a given material may vary based on temperature, pressure, oxygen concentration, and other factors.

The LFL and UFL generally define or bound a flammability envelope between the limits that refers to fuel concentrations relative to oxygen that would allow for combustion. In other words, a mixture that is within the “flammability envelope” would ignite and burn in the presence of an ignition source. A wider flammability envelope creates a higher risk of combustion (and thus a higher safety risk) because there are a broader range of conditions under which a mixture of fuel and oxygen could ignite or combust. A narrower flammability envelope is the inverse – there is a narrower range of conditions at which combustion could occur. The flammability envelope of propylene is relatively wide at the temperatures mentioned above and the flammability envelope increases or widens with increasing temperatures, which thereby increases the risk of ignition of the mixture and an explosion at a processing plant. Specifically, the propylene and air mixture may have a LFL of around 2% and an UFL of around 11.1%.

As noted above, conventional processes for producing acrylic acid result in the mixture passing through the flammability envelope. The hot air stream may be pre-heated by the heat of compression of air from ambient conditions to desired pressure, which introduces variability based on ambient conditions, humidity, compressor performance, and other factors. If the preheat of the air is not carefully controlled, dangerous conditions for ignition of the mixture can be created, which increases the likelihood of an explosion at a processing plant. Thus, conventional processes for production of acrylic acid have significant safety concerns.

It would therefore be desirable to have systems and methods for producing acrylic acid that overcome the above and other deficiencies and drawbacks of known systems and methods.

### BRIEF SUMMARY

The present disclosure contemplates mixing propylene, without superheating, with air, steam, and optionally recycle gas. In other words, the disclosure provides systems and methods for producing acrylic acid that do not involve superheating the propylene prior to mixing with the other streams. This will result in the mixture having a lower temperature and thereby narrow the flammability envelope and reduce the risk of the mixture being in the flammability envelope during processing. After mixing, the reaction mixture can then be suitably heated to the reaction temperature.

In conventional solutions, the focus has been on ensuring that the reaction mixture is outside of the flammability envelope where possible, while still providing the necessary heat to allow the initiation of the oxidation reactions. When propylene is mixed with hot air, the mixing will pass through the flammability envelope. In the present disclosure, mixing is done at a reduced temperature. It is well known that at lower temperatures, the flammability envelope is less wide or is narrower. As a result, the concepts of the disclosure provide for mixing at a narrower flammability envelope such that the mixture spends less time in a potentially combustible condition, thereby improving safety.

The disclosure also contemplates using various media (preferably steam) to more precisely control the temperature of the reaction mixture after mixing and ensure that the temperature does not increase beyond the desired temperature range to further improve safety. Specifically, a heat exchanger may be included downstream of the mixer relative to a direction of fluid flow through the mixer which uses steam as the heating medium. Using steam as the heating medium allows for predictable and reliable control of the temperature of the reactor feed and ensures the same does not exceed a selected temperature. Thus, the further heat exchanger reduces the risk of a process excursion by the compressor and further improves safety.

Additional features, benefits, and advantages of the concepts of the disclosure are provided in the following detailed description. Accordingly, the disclosure is not limited by the foregoing summary.

### BRIEF DESCRIPTION OF THE SEVERAL VIEWS OF THE DRAWINGS

FIG. 1 is a schematic view of a conventional system for producing acrylic acid.

FIG. 2 is a schematic view of an implementation of a system for producing acrylic acid according to the present disclosure.

FIG. 3 is a schematic view of a further implementation of a system for producing acrylic acid according to the present disclosure.

FIG. 4 is a schematic view of a further implementation of a system for producing acrylic acid according to the present disclosure.

### DETAILED DESCRIPTION

Persons of ordinary skill in the relevant art will understand that the present disclosure is illustrative only and not in any way limiting. Other implementations of the presently disclosed systems and methods readily suggest themselves to such skilled persons having the assistance of this disclosure.

Each of the features and teachings disclosed herein can be utilized separately or in conjunction with other features and teachings to provide devices, systems, and methods for the production of acrylic acid. Representative examples utilizing many of these additional features and teachings, both separately and in combination, are described in further detail with reference to the attached Figures. This detailed description is merely intended to teach a person of skill in the art further details for practicing aspects of the present teachings and is not intended to limit the scope of the claims. Therefore, combinations of features disclosed in the detailed description may not be necessary to practice the teachings in the broadest sense and are instead taught merely to describe particularly representative examples of the present teachings.

Moreover, the various features of the representative examples and the dependent claims may be combined in ways that are not specifically and explicitly enumerated to provide additional useful implementations of the present teachings. It is also expressly noted that all value ranges or indications of groups of entities disclose every possible intermediate value or intermediate entity for the purpose of original disclosure.

The present disclosure is generally directed to systems and methods for producing acrylic acid that improve the overall safety of the process by mixing the propylene at a lower temperature to narrow the flammability envelope while also strictly controlling temperature after mixing to avoid process excursions that may lead to unsafe conditions. In other words, the concepts of the disclosure overcome the deficiencies and drawbacks of known systems through temperature control before, during, and/or after mixing. Additional features, benefits, and advantages of the concepts of the disclosure are provided below.

In the following description, the word “line” is construed broadly to mean any enclosed structure capable of carrying or conveying a fluid (i.e., liquid or gas) and includes, but is not limited to, pipes, conduits, and the like as a single piece or multiple pieces interconnected with fittings, brackets, fasteners, etc. For example, a “line” may be a single pipe connected between other structures, as noted, or may include multiple sections of pipe interconnected to each other with fittings. Further, the word “coupled” means joined or linked together and is construed to include intervening structures. For example, a pipe coupled to a heat exchanger allows for intervening structures to be between the pipe and the heat exchanger, such as a compressor, unless the word “directly” is used. A “direct coupling” or “directly coupled” for purposes of this disclosure refers to an uninterrupted connection between components without intervening structures. In other words, a pipe directly coupled to a heat exchanger means that there are no additional structures or components between the pipe and the heat exchanger except for fittings, fasteners, and the like that establish the connection. As a result, a pipe that is directly coupled to a heat exchanger provides a fluid path that feeds into the heat exchanger without first passing the fluid through any intervening structures, such as compressors, reactors, and the like.

“Upstream” and “downstream” are relative terms based on a direction of fluid flow through the system or components of the system, such that a heat exchanger upstream of a mixer is a heat exchanger that is located earlier in the flow path of fluid through the system than the mixer or located closer to a source of the fluid flow in the system than the mixer. A mixer downstream of a heat exchanger is the opposite of the provided example for upstream. “Direct fluid communication” is similar to the definition of “directly coupled” provided above except in the context of ability to convey a fluid. Thus, two components that are in direct fluid communication with each other convey a fluid from the first component to the second component without passing the fluid through intervening components or structures, such as additional heat exchangers and the like, other than lines, pipes, conduits, etc. that establish the fluid connection.

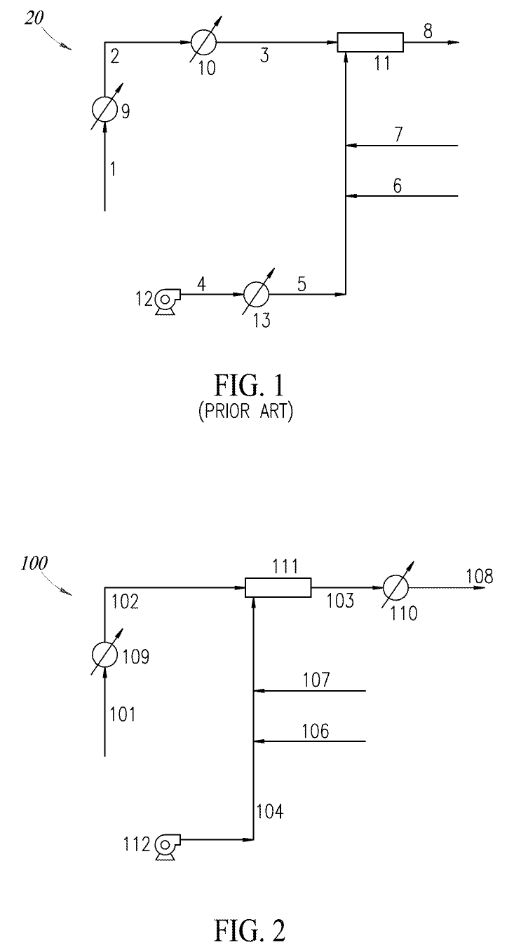

FIG. 1 is a schematic representation of a conventional system 20 and associated methods for producing acrylic acid that is provided for additional background regarding the concepts of the disclosure. In system 20, a feed of propylene enters the production facility or system 20 through line 1. The propylene feed may be liquid, in which case, it is vaporized in heat exchanger 9. Vaporized propylene is then fed to a superheater 10 along line 2, which may be a further heat exchanger. The vaporized propylene is superheated at the superheater 10 to a temperature in the range of 50-75°C and output as a superheated, vaporized propylene stream along line 3 to a mixer 11 or mixing device 11.

Air is compressed in compressor 12 and output through line 4. The air may or may not be pre-heated upstream of the compressor 12 depending on the application. The compressed air is provided along line 4 from the compressor 12 to heat exchanger 13, where it is heated. The heat exchanger 13 may be a start-up heater to increase the temperature of the compressed air from compressor 12 during start-up of the system 20. Once the system 20 is running at normal operating conditions (i.e., after the start-up period), the heat of compression from the compressor 12 may act as the preheat and/or heater for the air and the heat exchanger 13 may not be utilized unless there is a need for additional heat input to the compressed air stream. The heated air stream from the compressor 12 and/or the heat exchanger 13 is provided via line 5 to the mixer 11. Along line 5, steam and recycle gas are mixed with the heated air stream from the compressor 12 and/or the heat exchanger 13. Specifically, steam may be introduced along line 6 and recycle gas may be introduced along line 7. In various implementations, introduction of the recycle gas and/or steam is optional and omitted. The combined air, steam, and optional recycle gas mixture is in a temperature range of 120-220°C, and more preferably in the range 160-200°C. The mixture provided along line 5 is then mixed with superheated propylene from line 3 in the mixing device 11. The resulting mixture is fed to the reactor or reactors (not shown) along line 8 for conversion to acrylic acid. The mixture in line 8 (i.e., propylene, air, steam and optionally recycle gas) is in a temperature range of 140-180°C, which may be a reaction temperature for the mixture such that additional heating or cooling downstream of the mixer 11 is not needed. In other words, the temperature of the mixture leaving the mixer 11 along line 8 is sufficient for reaction without additional heat input and/or cooling.

While the two streams from lines 3, 5 are mixed at the mixing device 11, the propylene composition passes through the flammability envelope and the final composition has a temperature above the UFL. Above the UFL, there is not enough oxygen for combustion such that the mixture in line 8 has lower safety risk once it reaches the reaction temperature above the UFL. Mixing the propylene with the air (i.e., mixing the streams from lines 3, 5) is risky because propylene is highly flammable. To reach the reaction temperature, the mixture must pass through the flammability envelope which creates the risk of an explosion. It is well known that the width of the flammability envelope increases with temperature. Therefore, operating the mixing device 11 at a higher temperature such that the heated mixture 8 is suitable for reaction directly following the mixer 11 widens the flammability envelope and increases the risk of combustion and an explosion, as further explained herein.

Further, the heat of compression at the compressor 12 is the primary source of preheating the mixture at the mixer 11. This further increases risk because of a number of variations that may impact the final temperature of the mixture in line 5 into the mixer 11. Examples include fluctuation in ambient conditions (for example in temperature or relative humidity between day/night or summer/winter operation), variations in system pressure drop due to fouling or catalyst performance differences, or any mechanical issue with the compressor 12. Such process fluctuations could increase the mixed stream temperature in line 5 such that the composition lies within the flammability envelope, thus offering no further control or mitigation against a hazardous condition, and leading to uncontrolled conditions that significantly increase the likelihood of combustion of the mixture and an explosion at a processing plant. Conventional systems such as system 20 do not have appropriate safeguards or controls in place to prevent the development of these dangerous conditions.

FIG. 2 is a schematic view of a system 100 and related methods to produce acrylic acid according to the present disclosure. In the system 100, the mixer is operated at a lower temperature, which results in a narrower flammability envelope and thereby improved safety. Preferably, the system 100 is operated such that mixing occurs below the LFL and the mixture is heated to the reaction temperature after mixing in a carefully controlled process to reduce the risk of combustion and/or an explosion in the system 100. In other words, the system 100 provides two separate aspects that are each effective to improve the safety of the system 100. When these two aspects are combined, there is a significant resulting safety benefit.

In FIG. 2, a propylene feed enters the system 100 through line 101. If the propylene feed 101 is liquid, then it is vaporized in heat exchanger 109 and leaves as vaporized propylene along line 102 to mixer 111 (or mixing device 111). In an implementation, the vaporized propylene is provided directly from the heat exchanger 109 to the mixer 111 (i.e., the heat exchanger 109, line 102, and mixer 111 are directly connected to each other or in direct fluid communication with each other via line 102). As such, the system 100 omits the superheater 10 from system 20 and does not superheat the propylene prior to mixing in order to provide the propylene to the mixer 111 at a lower temperature. In an implementation, the temperature of the vaporized propylene in line 102 provided from the heat exchanger 109 directly to the mixer 111 is in a range between 0-10°C and is preferably about 3°C. Air is compressed in compressor 112 to a desirable pressure and is fed through line 104. The compressed air in line 104 is mixed with steam and recycle gas through pipes or lines 106 and 107, respectively. The lines 106, 107 may also be referred to herein as inputs 106, 107 and/or input lines 106, 107. In an implementation, mixing with recycle gas through line 107 is optional and may be omitted. The combined air, steam and recycle gas mixture is in a temperature range of 160-200°C. The air, steam and recycle gas mixture is then provided to the mixer 111 and mixed with the propylene vapors from line 102.

In an implementation, the line 104 is directly connected between the compressor 112 and the mixer 111 with the input lines 106, 107 adding additional material to the mixture rather than being intervening structures. Thus, the system 100 also omits the heat exchanger 13 relative to system 20. As will be further described below, the reaction mixture is heated after or downstream of the mixer 111 such that start-up heating of the air in line 104 is not needed. Instead, the heating after the mixer 111 will account for variations in the temperature of the air and thus the mixture at the mixer 111 if the system 100 is in a start-up condition. To improve the safety of the process, it is also preferable that the mixture at the mixer 111 is at a reduced temperature to allow margin for the process variations discussed above with respect to system 20. Thus, omitting the heat exchanger 13 and starting the process with a colder mixture or colder compressed air stream enables, at least in part, the benefits described herein.

In sum, the propylene in line 102 is not superheated prior to mixing and there is no additional heat input between the compressor 112 and the mixer 111. This results in a lower temperature of the mixture at the mixer 111, which narrows the flammability envelope. In an implementation, the mixture of propylene, air, steam, and optionally recycle gas at the mixer 111 is in the range of 130-180°C, more preferably between 130-150°C, and more preferably about 140-150°C. The disclosure of these ranges also provides support for a range with any selected value within the stated broad ranges. Thus, for example, a temperature range of 140-180°C is contemplated and supported, as is a range of 135-165°C, etc. Thus, changing known processes to remove the superheating of the propylene and avoid additional heat input between the compressor 112 and the mixer 111 results in a temperature of the mixture at the mixer 111 that is below the preferred temperature range at the mixer 11 in system 20, or in other words, the mixture at mixer 111 is below the preferred reaction temperature range of the mixture, which narrows the flammability envelope and improves safety. Accordingly, one aspect of the disclosure is achieving improved safety by conducting mixing at a temperature below a preferred reaction temperature of the mixture. In addition to this benefit, the system 100 further provides for control and mitigation against hazardous conditions, as further explained below.

After mixing at the mixer 111, the mixture leaving along line 103 is subsequently heated in heat exchanger 110 to the reaction temperature (i.e., 140-180°C) before being provided along line 108 to one or more reactors (not shown) downstream of the heat exchanger 110. In other words, according to the concepts of the disclosure, the mixture from the mixer 111 is heated to the reaction temperature after or downstream of the mixer 111 relative to a direction of fluid flow through the mixer 111 instead of the mixture exiting the mixer 11 at the reaction temperature as in system 20. This is a significant difference because mixing occurs at the mixer 111 in system 100 at a lower temperature than in system 20, while also allowing for final adjustment of the reactor feed temperature at line 108 via heat exchanger 110 to account for process variations. In other words, the concepts of the disclosure provide for control and mitigation against hazardous conditions with heat exchanger 110. As a result, the flammability envelope is narrowed, the risk of ignition is reduced, and the safety of the process is improved.

The heat exchanger 110 may use a variety of different heating mediums to accomplish the heating of the reaction mixture in line 103. Such heating mediums include, but are not limited to, hot oil, steam, electricity, and other process fluids (i.e., recycle gas, compressed air, etc.) in the system 100, although any other heating medium is contemplated. In a preferred implementation, the heating medium is steam meaning that the reaction mixture from line 103 is heated against steam to produce the heated reaction mixture output from the heat exchanger 110 to the reactor(s) along line 108. Steam is preferred because it is readily available in the overall process for producing acrylic acid including system 100, but also because steam allows for careful temperature control, thus avoiding process excursions that can create dangerous conditions and increase risk. By using steam as the heating medium, the condensate temperature can be controlled using a variety of techniques to ensure that the reaction mixture leaving the heat exchanger 110 in line 108 does not exceed a selected temperature, such as between 140-180°C. For example, the pressure of condensing steam can be controlled to ensure that the temperature of the reaction feed in line 108 does not increased beyond the selected temperature.

Further, and as noted above, the addition of heat exchanger 110 downstream of the mixer 111 allows for omission of the heat exchanger 13 because the heat exchanger 110 can account for variations in temperature of the mixture in line 103 from the mixer 111. Moreover, careful control of the temperature of the reaction mixture via heat exchanger 110 significantly reduces the risk of dangerous conditions created by the heat of compression from the compressor 112 because even if there is an issue with the compressor 112, the heat exchanger 110 can heat (or cool) the reaction mixture as needed to control and/or prevent the occurrence of dangerous conditions.

Thus, in sum, in the conventional configuration shown in FIG. 1, heat of compression of the air from ambient conditions to the desired pressure at compressor 12 is the source of pre-heat of the air stream. This introduces variability based on ambient conditions, humidity, process pressure drop, and compressor performance. If the preheat via compressor 12 is not carefully controlled, this can lead to dangerous conditions at the mixer 11. In the system 100, heat exchanger 110 acts as the final heat source to bring the reactor feed gas in line 103 to reaction temperature, thereby reducing the impact of ambient conditions and leading to a more stable temperature of reactor feed gas in line 108 that leaves the heat exchanger 110. The conventional configuration shown in FIG. 1 also includes the superheater 10 to heat the propylene stream 2 for mixing such that the resultant mixture leaves mixer 11 at reaction temperature along line 8. This is a further source of risk in the system. In the system 100, the superheater 10 is omitted so that mixing occurs at a reduced temperature and narrower flammability envelope at the mixer 111. Thus, in system 100, the propylene vapors transition through a smaller flammability envelope, thus resulting in improved safety. As a further benefit, omission of the superheater 10 in the system 100 reduces equipment count and cost.

The disclosure also contemplates using the heat exchanger 13 as the heat exchanger 110, or in other words, retrofitting an existing system such as system 20 by removing the superheater 10 and relocating and/or repurposing the heat exchanger 13 as the heat exchanger 110 in order to provide the benefits described herein to existing systems as well as new systems. While it is preferred that the above aspects to improve the safety of the production of acrylic acid be used in combination to provide all of the benefits described herein, the same is not necessarily required. In some implementations, the system 100 may be the same as system 20 except for the omission or idling of only the superheater 10 to lower the temperature at the mixer 111 and provide some of the benefits described herein. Further, the system 100 may generally be the same as system 20, except the heat exchanger 110 is provided downstream of the mixer 110 in order to assist with temperature control.

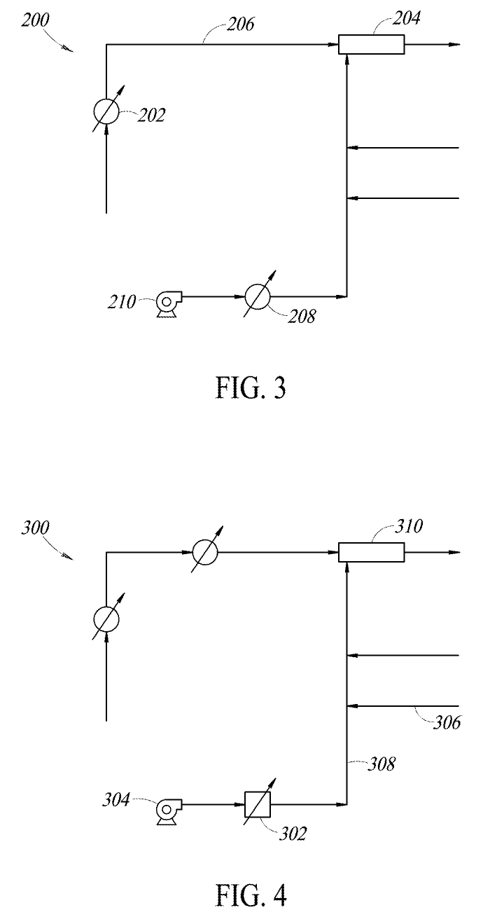

For example, FIG. 3 is a non-limiting example of a system 200 where the superheater 10 is omitted. Instead of the superheater 10, the system 200 includes a heat exchanger 202 in direct fluid communication with a mixer 204 along line 206. The heat exchanger 202 receives and vaporizes the propylene as needed. The vaporized propylene stream is then provided to the mixer 204 without further heating and/or superheating. The removal of the superheater may lower the temperature of the propylene and/or the resulting reactor feed output from the mixer 204 by about 20°C to provide some of the benefits described herein, such as mixing at a lower temperature to narrow the flammability envelope. In some implementations of the system 200, a further heat input downstream of the mixer 204, such as heat exchanger 110, may be needed to heat the resulting reactor feed to reaction temperature. Alternatively, heat exchanger 208 downstream of compressor 210 may be used to provide additional heat input, but this may reduce or eliminate the benefit of omitting the superheater because the mixing at the mixer 204 will occur at a similar temperature as in system 20 even if the components provided to the mixture are at different temperatures (i.e., propylene at lower temperature and air, steam, and recycle gas mixture at higher temperature).

FIG. 4 is a schematic view of a further implementation of a system 300 according to the present disclosure. The system 300 may be similar to others discussed herein, except a cooler 302 is provided downstream of a compressor 304 and upstream of additional input lines 306. In an implementation, the cooler 304 may be located anywhere along line 308 from the compressor 304 to a mixer 310, such as downstream of the input lines 306 but upstream of the mixer 310. The cooler 302 may utilize any available cooling medium, such as water, other process fluids in the system 300, and others, to provide temperature control of the air output from the compressor 304. Thus, the cooler 302 can be utilized to avoid process excursions that result from the heat of compression at the compressor 304 and provide some of the benefits described herein. The downside to this approach is that the cooler 302 utilizes significant energy, which negatively impacts the overall efficiency of the system 300. Thus, the system 300 may be a less efficient solution to provide some of the advantages described herein relative to system 100. In an implementation, the system includes a heat exchanger either upstream or downstream of the cooler 302.

In yet a further implementation of the systems 100, 200, 300 described herein, the heat exchanger 110 downstream of the mixer 111, 204, 310 can be designed or configured to provide cooling while shutting down the reactor(s) as a further safety benefit. The disclosure also includes related methods to the systems described herein, such as flowing the described fluids through the component parts of the system and heating and/or processing the same as described above.

The above description of illustrated implementations, including what is described in the Abstract, is not intended to be exhaustive or to limit the implementations to the precise forms disclosed. Although specific implementations and examples are described herein for illustrative purposes, various equivalent modifications can be made without departing from the spirit and scope of the disclosure, as will be recognized by those skilled in the relevant art. The teachings provided herein of the various implementations can be applied outside of the acrylic acid context, and are not limited to the example systems, methods, and devices for production of acrylic acid generally described above.

Many of the methods described herein can be performed with variations. For example, many of the methods may include additional acts, omit some acts, and/or perform acts in a different order than as illustrated or described.

In the above description, certain specific details are set forth in order to provide a thorough understanding of various implementations of the disclosure. However, one skilled in the art will understand that the disclosure may be practiced without these specific details. In other instances, well-known structures associated with devices, systems, and methods for production of acrylic acid have not been described in detail to avoid unnecessarily obscuring the descriptions of the implementations of the present disclosure.

Throughout the specification, claims, and drawings, the following terms take the meaning explicitly associated herein, unless the context clearly dictates otherwise. As used throughout this document, including the claims, the singular form “a”, “an”, and “the” include plural references unless indicated otherwise. Any of the features and elements described herein may be singular, e.g., a mixer may refer to one mixer. The terms “include” and “comprise,” as well as derivatives thereof, mean inclusion without limitation. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Other definitions of certain words and phrases are provided throughout this disclosure.

The use of ordinals such as first, second, third, etc., does not necessarily imply a ranked sense of order, but rather may only distinguish between multiple instances of an act or a similar structure or material.

The term “herein” refers to the specification, claims, and drawings associated with the current application. The phrases “in one implementation,” “in another implementation,” “in various implementations,” “in some implementations,” “in other implementations,” and other derivatives thereof refer to one or more features, structures, functions, limitations, or characteristics of the present disclosure, and are not limited to the same or different implementations unless the context clearly dictates otherwise. As used herein, the term “or” is an inclusive “or” operator, and is equivalent to the phrases “A or B, or both” or “A or B or C, or any combination thereof,” and lists with additional elements are similarly treated.

Generally, unless otherwise indicated, the materials for making the invention and/or its components may be selected from appropriate materials such as composite materials, ceramics, plastics, metal, polymers, thermoplastics, elastomers, plastic compounds, steel, and the like, either alone or in any combination.

The terms “top,” “bottom,” “upper,” “lower,” “up,” “down,” “above,” “below,” “left,” “right,” and other like derivatives take their common meaning as directions or positional indicators, such as, for example, gravity pulls objects down and left refers to a direction that is to the west when facing north in a Cardinal direction scheme. These terms are not limiting with respect to the possible orientations explicitly disclosed, implicitly disclosed, or inherently disclosed in the present disclosure and unless the context clearly dictates otherwise, any of the aspects of the implementations of the disclosure can be arranged in any orientation.

As used herein, the term “substantially” is construed to include an ordinary error range or manufacturing tolerance due to slight differences and variations in manufacturing. Unless the context clearly dictates otherwise, relative terms such as “approximately,” “substantially,” and other derivatives, when used to describe a value, amount, quantity, or dimension, generally refer to a value, amount, quantity, or dimension that is within plus or minus 5% of the stated value, amount, quantity, or dimension. It is to be further understood that any specific dimensions of components or features provided herein are for illustrative purposes only with reference to the various implementations described herein, and as such, it is expressly contemplated in the present disclosure to include dimensions that are more or less than the dimensions stated, unless the context clearly dictates otherwise.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the breadth and scope of a disclosed implementation should not be limited by any of the above-described implementations, but should be defined only in accordance with the following claims and their equivalents.

1. A system, comprising:
a first heat exchanger configured to heat a propylene stream and output a vaporized propylene stream;
a compressor configured to compress an air stream and output a compressed air stream;
a mixer in fluid communication with the first heat exchanger and the compressor; and
a second heat exchanger in fluid communication with the mixer downstream of the mixer relative to a direction of flow through the mixer,
wherein the mixer is configured to mix the vaporized propylene stream and the compressed air stream and output a reaction feed, and
wherein the second heat exchanger is configured to heat the reaction feed to a reaction temperature.

2. The system of claim 1, further comprising:
at least one input line in fluid communication with the compressor and the mixer,
wherein the at least one input line is downstream of the compressor relative to a direction of flow through the compressor.

3. The system of claim 2, wherein the at least one input line is configured to introduce steam or recycle gas, or both, to the compressed air stream.

4. The system of claim 1, wherein a heating medium of the second heat exchanger is steam.

5. The system of claim 1, further comprising:
a cooler between the compressor and the mixer, the cooler configured to control a temperature of the compressed air stream.

6. The system of claim 1, wherein a temperature of the reaction feed output from the mixer is less than the reaction temperature.

7. The system of claim 1, wherein a temperature of the reactor feed is less than 140°C, or
wherein a temperature of the vaporized propylene output from the first exchanger is in a range of 0-10°C.

8. The system of claim 1, wherein the first heat exchanger is in direct fluid communication with the mixer, or
wherein the second heat exchanger is in direct fluid communication with the mixer.

9. A system, comprising:
a mixer; and
a heat exchanger in direct fluid communication with the mixer downstream of the mixer relative to a direction of flow through the mixer,
wherein the mixer is configured to mix a vaporized propylene stream and an air stream and output a reaction feed at a temperature lower than a reaction temperature of the reaction feed, and
wherein the second heat exchanger is configured to heat the reaction feed to the reaction temperature.

10. The system of claim 9, further comprising:
a compressor configured to provide the air stream to the mixer.

11. The system of claim 10, further comprising:
a further heat exchanger upstream of the mixer relative to the direction of flow through the mixer, the further heat exchanger in direct fluid communication and configured to provide the vaporized propylene stream to the mixer.

12. The system of claim 9, wherein a temperature of the reaction feed output from the mixer is in a range of 130-150°C.

13. The system of claim 9, wherein a temperature of the vaporized propylene stream is in a range of 0-10°C.

14. The system of claim 9, wherein a heating medium of the second heat exchanger is at least one of steam, hot oil, electricity, and process fluid.

15. A method, comprising:
heating a propylene stream at a first heat exchanger, including outputting a vaporized propylene stream from the first heat exchanger;
compressing an air stream at a compressor, including outputting a compressed air stream;
mixing the vaporized propylene stream and the compressed air stream at a mixer and outputting a reaction feed from the mixer; and
heating the reaction feed to a reaction temperature at a second heat exchanger downstream of the mixer relative to a direction of flow through the mixer.

16. The method of claim 15, further comprising:
mixing the compressed air stream with steam or recycle gas, or both, upstream of the mixer relative to the direction of flow through the mixer.

17. The method of claim 15, wherein heating the reaction feed includes heating the reaction feed against steam as a heating medium at the second heat exchanger.

18. The method of claim 15, wherein outputting the vaporized propylene stream from the first heat exchanger includes providing the vaporized propylene stream directly from the first heat exchanger to the mixer.

19. The method of claim 15, wherein heating the propylene stream at the first heat exchanger includes heating the propylene stream to a temperature of 0-10°C, and
wherein outputting the reaction feed from the mixer includes outputting the reaction feed from the mixer at a temperature of 140-180°C.

20. The method of claim 15, wherein outputting the reaction feed from the mixer includes providing the reaction feed directly from the mixer to the second heat exchanger.
